(19) United States
(12) Patent Application Publication
(10) Pub. No.: US 2026/0327551 A1
(43) Pub. Date: Oct. 22, 2026

(54) Sperm Separation System

(71) Applicants: Theodore Leland Tollner, Wilton, CA (US); Corey Burke, Oviedo, FL (US); Reprovantage Laboratories, Wilton, CA (US)

(72) Inventor: Theodore Leland Tollner, Wilton, CA (US)

(21) Appl. No.: 19/130,375

(22) PCT Filed: Nov. 15, 2023
(86) PCT No.: PCT/US2023/079869

§ 371 Date: May 15, 2025

Related U.S. Application Data
(60) Provisional application No. 63/425,566, filed on Nov. 15, 2022.

(51) Int. Cl. C12M 1/00; C12M 1/26; C12M 3/00

(52) U.S. Cl. CPC C12M 47/02; C12M 21/06; C12M 33/14

(57) ABSTRACT

A device for the collection of motile sperm is disclosed, the device including a column having a top, a bottom, and a wall, the wall including a mesh layer and a membrane layer, an inlet disposed at the top of the column, and a nosepiece disposed at the bottom of the column.

### CROSS REFERENCE TO RELATED APPLICATION

This application claims the benefit of U.S. Provisional Application No. 63/425,566, filed on Nov. 15, 2022, the entire disclosure of which is hereby incorporated herein by reference.

### FIELD OF THE INVENTION

The present application provides a device for separating motile sperm of high quality from dead, dying, and immature sperm as well as non-spermic cells commonly found in semen. The device is used to process an entire human semen specimen without the need for centrifugation or manipulation with other equipment, special handling or training, or customized collection containers or culture ware. The disclosure provides a device and a method for efficient preparation of sperm for therapeutic use. In some embodiments, the device and method described herein separate motile sperm from cryoprotectants following warming of frozen specimens. In some embodiments, the device can be used for processing of sperm from semen of livestock and for purposes of sperm biology research.

### BACKGROUND

According to the National ART (assisted reproductive technology) Summary published by the CDC for in vitro fertilization (IVF) procedures in 2019, over 281,000 treatment cycles required the use of processed sperm. Many more sperm specimens are processed annually for intrauterine insemination (IUI) although the number of IUI cycles are unknown in the US for lack of a registry for artificial insemination procedures. Assuming a similar trend exist as in in European countries which report IUI outcomes (Ferratti et al., 2013), some 28% of total infertility cycles, or well over 100,000 IUI cycles may be performed annually in the United States. Whether specimens are from partners or donors, fresh or frozen-thawed, the vast majority of sperm samples (we estimate approximately 400,000 annually) must be processed prior to use in IVF or IUI.

There are numerous techniques for processing sperm for fertility treatment. The general strategy of most processing methods is to separate the more healthy, motile sperm from the dead and dying sperm, white cells, and other potentially deleterious components of semen. The higher quality motile sperm derived from the separation process are then used for IUI or IVF. Many processing methods utilize one or more cycles of centrifugation to concentrate and remove sperm from seminal plasma. The commonly used “density gradient wash” system involves layering semen on top of a column of colloidal solution comprised of either polyvinylpyrrolidone-coated or silane-coated silica particles in a standard 15 cc centrifuge tube (Henkel and Schill, 2003; for review). Upon centrifugation, motile sperm preferentially move through the density gradient solutions (DGS) and are concentrated in a diffuse layer at the bottom of the tube. These more motile sperm are recovered and “washed” free of gradient solution with culture medium via another round or two of centrifugation. The end result is a relatively high yield of mostly motile sperm with little to no contamination with seminal plasma. Typically, 30-40% of the total number of motile sperm in a semen specimen are recovered following gradient wash with 75%-90% of the recovered sperm having vigorous forward motion, or “progressive motility” (World Health Organization, 2021). The draw-back of the gradient wash, as well as other centrifuge-based cell washing techniques, is that centrifugation induces the generation of reactive oxygen species (ROS; Shekarriz et al., 1995; Mckinney et al., 1996) which can damage sperm structures including DNA (González-Marín et al., 2012; Aitken et al., 2022).

In light of the well-reported impact of sperm DNA fragmentation on fertility treatment outcomes (Agarwal et al., 2022; for review; Marinaro and Schlegel, 2023; for review), many laboratory practitioners have opted for less forceful or more passive sperm selection techniques to minimize the generation of ROS. In a standard “Swim Up”, culture medium is carefully layered on top of semen, or in some cases on top of a layer of sperm concentrated from semen by a single cycle of relatively “gentle” (low g-force) centrifugation. Some of the motile sperm will swim upward into the medium above. A portion of Swim Up medium is then sampled, taking care not to disturb the interface between the lower sperm/semen layer and the medium (Henkel and Schill, 2003; for review).

Similar in outcomes to the standard Swim Up, and recently introduced to marketplace, the Zymot® “Multi”, a resinous cartridge or “chip” (Demirci et al. 2016; Asghar et al., 2014), and the Lenshooke® “CA0”, a “Dish” (Hsu et al., 2023), are sperm sorting devices which operate on a similar principles. Sperm from semen dispensed into a lower chamber swim up though a microporous membrane into an upper chamber of culture medium (Demirci et al. 2016; Asghar et al., 2014; Hsu et al., 2023). The interface between semen and medium is well-secured with the microporous membrane, facilitating easy recovery of sperm from the upper chamber. The advantage of the Swim Up and the sorting devices is they generally result in the separation (or sorting) of a highly motile population of sperm, over 90% of which have progressive motility. The disadvantage of these systems is they are inefficient, resulting in low total numbers of motile sperm. Reports are lacking on the efficiency of a single Swim Up column, but multiple columns set up in parallel to maximize sperm recoveries tend to yield fewer than 20% of the total number of motile cells found in a semen specimen (World Health Organization, 2021). A Zymot® “Multi” chip yields on average 24% of the motile sperm found in a volume of semen loaded into the device (Demirci et al. 2016; Asghar et al., 2014). A single chip, however, can only accommodate approximately a third of the volume of an average ejaculate and, therefore, only 8% of the total numbers of motile sperm found in the average semen specimen are recovered. Efficiency of recovery of motile sperm with the Lenshooke® “CA0” has not been reported but like the Zymot® “Multi” only a fraction of a semen specimen can be processed with a single device (Hsu et al., 2023). It is not infrequent that the numbers of motile sperm recovered from these passive separation methods are suboptimal for IUI (Samuel et al., 2018). As with the Swim Up, multiple chips and dishes can be processed in parallel to improve yield, but this is more time-consuming and the added expense may be difficult for some programs to justify.

At present, the infertility market lacks a passive, low-cost system of separating sperm that is both easy to use and results in a high yield of motile cells. Maximizing yield of motile sperm while minimizing sperm damage is especially important for men who have low sperm counts or who have unexplained infertility (sperm appear normal but only a small fraction of sperm in a semen specimen are fully functional). The yield of high-quality sperm following processing can be the difference between success and failure with some treatments for infertility.

### SUMMARY

In some embodiments, a Sperm Separation System (SSS) device is disclosed, the device including a column having a top, a bottom, a wall having a mesh layer and a membrane layer, an inlet disposed at the top of the column, and a nosepiece disposed at the bottom of the column. In some embodiments, the device is configured to receive a fresh or frozen-thawed semen specimen (FIG. 1). Once loaded with semen, the SSS device may be inserted into one of any number of culture tube types used routinely in IVF and Andrology laboratories for sperm processing and incubation.

In some embodiments, the mesh layer is configured to structurally support the membrane layer.

In some embodiments, the membrane layer includes a plurality of micropores.

In some embodiments, the plurality of micropores include micropores having a size of at least 1 micrometer in diameter.

In some embodiments, the plurality of micropores includes micropores having a size of less than 10 micrometers in diameter.

In some embodiments, the membrane layer is configured to restrict non-motile sperm, white cells, and other round cells.

In some embodiments, the inlet includes a loading ring, the loading ring configured to receive liquid specimens.

In some embodiments, the membrane layer includes PVP-treated polycarbonate.

In some embodiments, the mesh layer is configured to restrict debris, clumps of agglutinated cells and other material, and semen coagulum from permeating the membrane layer.

In some embodiments, the mesh layer includes a plurality of openings, wherein the opening are at least 10 micrometers across and less than 100 micrometers across.

In some embodiments, the mesh layer includes stainless steel, the stainless steel configured to restrict bacteria from permeating the mesh layer.

In some embodiments, the wall includes a plurality of pleats or corrugations.

In some embodiments, a method for sample separation is disclosed, the method including receiving, by an inlet, a sample within a column, the column including a mesh layer and a membrane layer, depositing the column in a receptacle, the receptacle containing a culture medium, capturing, by the culture medium, a portion of the sample that exits the column through the mesh layer and the membrane layer, removing the column from the receptacle, and harvesting the captured portion of the sample from the culture medium.

In some embodiments, the sample used in the method for sample separation includes liquid containing sperm.

In some embodiments, the captured portion of the sample includes motile sperm.

In some embodiments, the method further includes filtering, by the membrane layer, and adhering and filtering, by the mesh layer, at least one of debris, agglutinated cells and other material, semen coagulum, non-motile sperm, bacteria, and other non-spermic cells from the sample.

In some embodiments, the method further includes depositing the column in a second receptacle, the second receptacle containing a second culture medium, and capturing, by the second culture medium, a second portion of the sample that exits the column through the mesh layer and the membrane layer.

In some embodiments, the receptacle includes a centrifuge tube, a cell-culture tube, a blood tube, a vial, or a cuvette.

In some embodiments, a method for assembling a device is disclosed, the method including aligning a mesh layer and a membrane layer, adhering the mesh layer to the membrane layer, forming a two-ply material, forming, via conforming the two-ply material, a column of a predetermined diameter and length, the column having a top and a bottom, and wherein the membrane layer of the two-ply material forms the outside of the column, and wherein the mesh layer of the two-ply material forms the inside of the column, coupling an input piece to the top of the column, and coupling a nosepiece to the bottom of the column.

In some embodiments, the device is cylindrical, and may be different diameters and heights, for use in different sized tubes. Two differently sized devices have been thoroughly tested, a larger one for use in 15 cc centrifuge tubes (FIG. 6A) and a smaller one for use with 1.5 ml cryovials and disposable mini-beakers or “dispo-beakers” (FIG. 6B).

In some embodiments, the device can be a square or triangular column for insertion into cuvettes that might also be used in conjunction with automated sperm analysis and other cellular assay systems.

In some embodiments, the device includes an inlet, the inlet having a loading ring (FIG. 2), the 2-ply microporous walls of the column (FIG. 5), and the nosepiece (FIGS. 3, 4). A semen specimen is loaded into the device via the loading ring, and fills the column, saturating the 2-ply walls but not overflowing or emitting through the micropores (FIG. 1A). The device is then inserted vertically, the nosepiece first, into a culture tube or centrifuge tube containing culture medium (FIG. 1B).

In such embodiments, culture medium may be configured to receive sperm that move out of the device (hereafter referred to as “Swim Out Sperm”) through the 2-ply microporous walls. The device is left in the upright tube for 15 to 60 minutes, during which time motile sperm accumulate in the medium (also referred to as “Swim Out Medium”) in the culture tube (FIG. 1B). The device may then be removed from the tube and discarded, or can be placed in another tube to collect additional Swim Out Sperm. In some embodiments, the Swim Out Sperm (sperm remaining in the Swim Out Medium in the culture tube; FIG. 1C) are of high quality, with average progressive motility of greater than 90% (FIG. 8B; FIG. 11A).

The methods and devices disclosed have several advantages over other passive sperm processing systems. First, a single SSS device (the larger cylindrical version sized for a standard 15 cc centrifuge tube; FIG. 1, FIG. 6A, B) can accommodate an ejaculate (semen specimen) of up to 4 ml in volume, which is greater than the average ejaculate volume of 2-3 ml (World Health Organization, 2021). Second, in some disclosed methods and devices, the Swim Out is performed into a single culture tube or centrifuge tube, instead of requiring additional devices, specialized chambers, or customized tubes, simplifying sperm processing and reducing costs. Third, disclosed methods and devices may be used with viscous specimens (non-liquefied semen, frozen-thawed specimens in egg yolk-based extenders or other diluents, etc), owing to the rigid mesh which constitutes the inner ply of the walls (the mesh restricts debris and coagulum from clogging the smaller pores of the polycarbonate membrane that comprises the outer ply). Fourth, the disclosed methods and devices may result in a higher yield of motile sperm, as compared to other processing methods, owing to, at least in part, the large surface area and vertical configuration of the separation surface (no matter what direction progressively motile sperm move they will eventually make contact with the column wall). In some embodiments, the disclosed devices and methods may produce an average yield exceeding 60% of the total motile sperm in an ejaculate from a single SSS device following a 30 to 60 min incubation (FIG. 8A, FIG. 10, FIG. 12A). Finally, the SSS is easier both in the set up and the recovery of sperm than either DGS washing systems or other passive separation methods, including the Zymot® “Multi” (the “Multi” requires the use of two tuberculin syringes, one for loading of semen and one for removal of the sorted sperm. Care must be taken with loading the semen specimen to ensure bubbles are not “injected” into the device which can be trapped against the separation membrane and obstruct sperm movement into the collection chamber).

### BRIEF DESCRIPTION OF THE FIGURES

FIGS. 1A-1C illustrate the operation of the SSS device, according to an example embodiment.

FIG. 2A is a schematic view of a loading ring from the bottom, according to an example embodiment. FIG. 2B is a schematic view of the loading ring of FIG. 2A from the side. And FIG. 2C illustrates a perspective view of the loading ring of FIG. 2A.

FIG. 3A is a schematic view of the top of the nosepiece, according to an example embodiment. FIG. 3B is a schematic view of the nosepiece in FIG. 3A from the side. FIG. 3C illustrates a perspective view of the nose piece in FIG. 3A.

FIGS. 4A-4B illustrate nosepieces, according to an example embodiment

FIGS. 5A-5E illustrates the components and assembly of the SSS device, according to an example embodiment.

FIGS. 6A-C illustrate the different sizes and designs of the SSS device that have been evaluated with human sperm;

FIGS. 7A-B is a table showing the biocompatibility of the SSS device and the materials from which it is comprised. FIG. 7A. Survival of DGS-washed sperm following 6-hour co-incubation at 37 °C with 1. a UV-cured adhesive, 2. stainless steel mesh, and 3. polycarbonate membrane compared to DGS-washed sperm (control) for each experiment. FIG. 7B. Survival of sperm 24 hours following 1. Swim Out of the large SSS device, 2. washing via DGS, and 3. sperm remaining in semen.

FIGS. 8A-B are graphs showing recovery of motile sperm from the small SSS device at 15 and 30 minutes

FIG. 9 is a graph showing recovery of motile sperm from large SSS devices at 30 and 60 minutes

FIG. 10 is a graph showing recovery (% R) of motile sperm from the large SSS device as it is moved through several centrifuge tubes containing fresh culture medium at defined time points; at 0, 30 min, and 60 min.

FIGS. 11A-D are graphs showing the quality of the sperm recovered from the SSS device as it is moved though several centrifuge tubes containing fresh culture medium at defined time points; at 0, 30 min, and 60 min. FIG. 11A manual counts of percent motility (% motile), FIG. 11B CASA measures of average sperm curvilinear velocity (VCL in um/sec), FIG. 11C sperm morphology (% normal forms; % N), and FIG. 12D the percentage of sperm with DNA fragmentation as detected with the TUNEL assay (% TUNEL Positive).

FIGS. 12A-E are graphs showing direct comparisons of DGS with SSS using the large device. The efficiency of recovery of motile sperm with the two processing methods is shown in FIG. 12A, the resulting sperm quality with each method is shown with FIG. 12B, counts of motile sperm (% Motile), FIG. 12C, CASA, FIG. 12D, hyaluronic Acid Gel Penetration Assay (HA penetration), and FIG. 12E survival following freezing and thawing.

### DETAILED DESCRIPTION

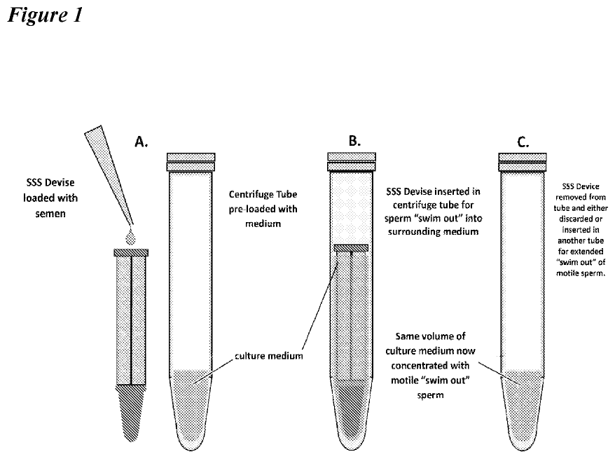

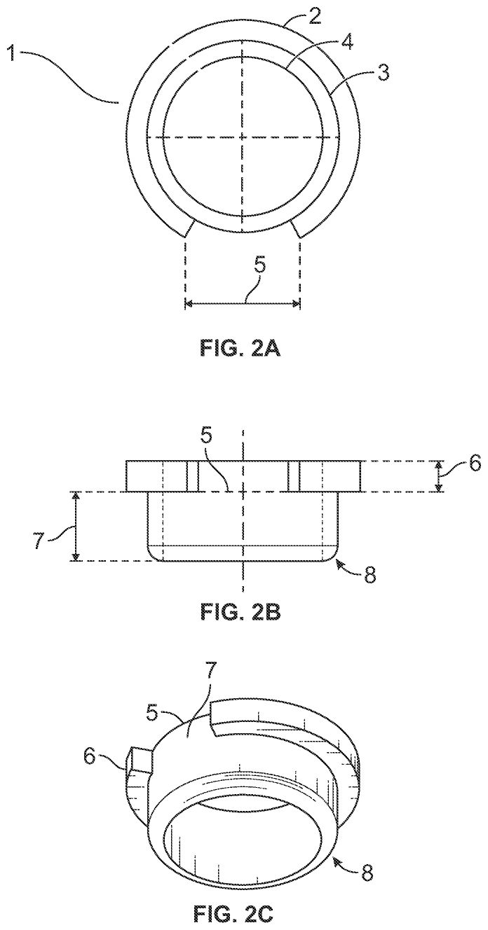

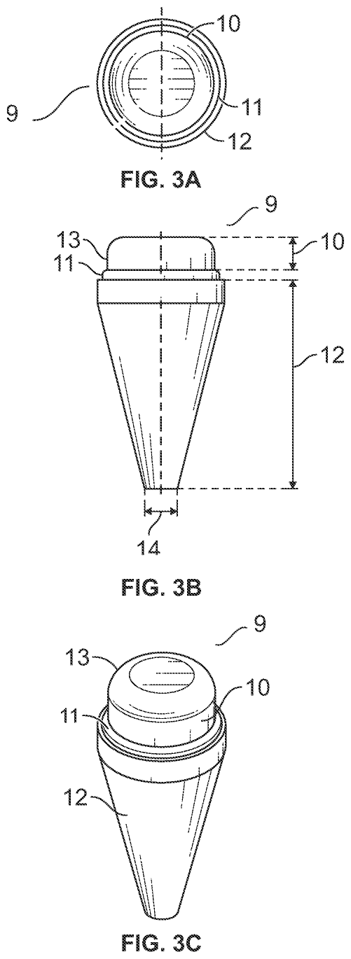

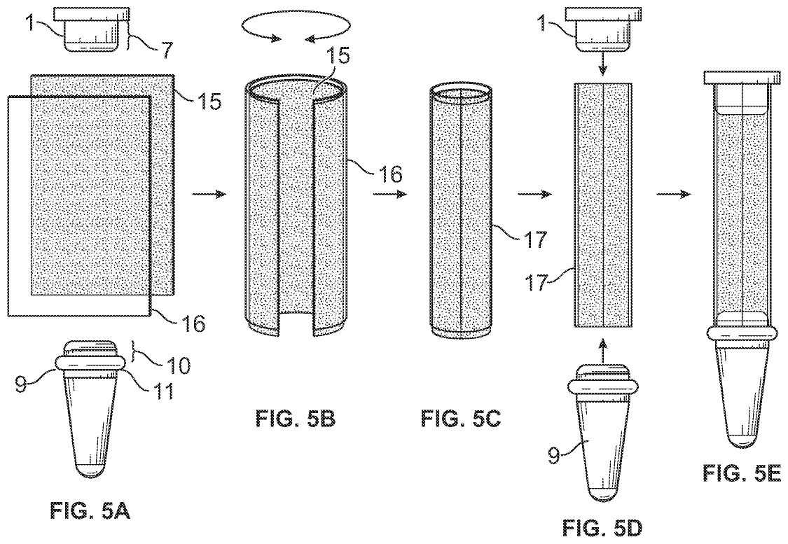

FIG. 1 illustrates the operation of the device in some embodiments. The device may loaded with semen or other sperm specimen. A standard centrifuge tube is loaded with 2 to 3 ml of culture medium (FIG. 1A). The SSS device may placed in the tube and the culture medium is displaced upwards and around the walls of the device (FIG. 1B). Motile sperm “swim out” from inside the device, through the walls comprised of mesh and microporous membrane, and into the culture medium surrounding the device (FIG. 1C). The device is removed leaving a high yield of motile sperm in the culture medium. In some embodiments, the SSS device is comprised of a loading piece or ring 1 (FIG. 2) positioned at the top, a nose piece 9 (FIG. 3) positioned at the bottom, both of which are connected to a column 17 with 2-ply walls (FIG. 5) positioned in the middle.

FIGS. 2A-C are schematic drawings of a loading piece or ring, according to an example embodiment. In FIG. 2A, a drawing of the loading ring 1 is shown from the bottom view, with an inner wall 4 of the loading ring 1, an outer wall 3 of the loading ring 1, and an outer most edge 2 of a rim of the loading ring 1. The rim is discontinuous, with a span or arc 5 of approximately 50 degrees where the rim is absent. In some embodiments, the arc 5 may include an angle greater or less than 50 degrees. In FIG. 2B, a side-view drawing of the loading ring shows a relative thickness 6 of the rim and a relative height of a stem 7. The stem 7 is the portion of the ring that may insert into an upper portion of a filtration column, such as filtration column 17 (FIG. 5C, D), with the rounded edge 8 of the stem 7 facilitating insertion into the filtration column. The 3-D perspective (FIG. 2C) shows clearly the absent span or arc 5 of the rim; this feature allows for an easy grip with forcepts for removing the device from a centrifuge tube. The span or arc 5 would also allow for attachment of a thin “finger strip” to assist with device insertion and removal.

FIGS. 3A-C are schematic drawings of the nosepiece 9, according to an example embodiment. In FIG. 3A, a view from the top shows an outer wall of a stem 10 of the nosepiece 9, an outer edge of a “fitting ridge” 11, and the widest point of a variable segment 12 of the nose-piece 9. In FIG. 3B, the side-view of the nosepiece 9 shows the relative heights of the stem 10, and the “fitting ridge” 11. The stem 10 inserts into a lower portion of a filtration column, such as filtration column 17 (FIG. 5C, D), and the rounded edge 13 of the stem 10 facilitates insertion. The “fitting ridge” 11 is similar in thickness to the 2-ply wall of the column and facilitates adherence with UV-catalyzed acrylate. The shape of the variable segment 12 (which includes the tip 14), can be changed without altering the fit of the nosepiece with the column. The 3-D perspective (FIG. 3C) shows clearly the circumferential “fitting ridge” 11 and that the nose piece is a closed.

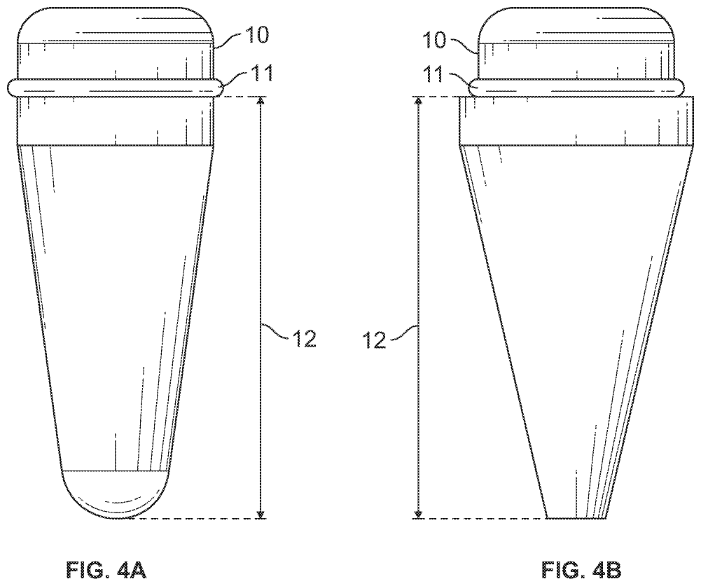

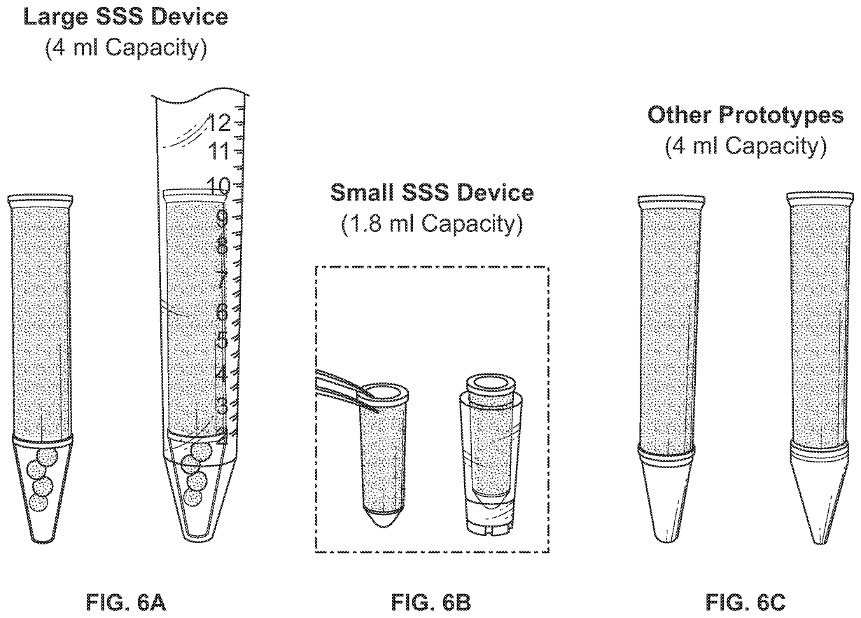

FIG. 4A-B shows the variable segment 12 of two prototypes, according to an example embodiment. SSS devices with a more slender, rounded nosepiece (FIG. 4A, FIG. 6C) were tested with 4 different makes of 15 ml conical centrifuge tubes. In each case, insertion of SSS devices with the more narrow nosepiece resulted in adequate displacement of culture medium around the device column walls. A wider and more tapered nosepiece (FIG. 4B, FIG. 6C) was designed for use in a specific brand of centrifuge tube with an uncommonly narrow conical tip. The dimensions of the stem 10 and the “fitting ridge” 11 for both nosepieces are the same, enabling attachment to device columns of the same diameter.

In some embodiments, a method for assembling a device is disclosed, the method including aligning a mesh layer and a membrane layer, adhering the mesh layer to the membrane layer, forming a 2-ply material, forming, via conforming the two-ply material, a column of a predetermined diameter and length, the column having a top and a bottom, the membrane layer forming the outside of the column and the mesh layer forming the inside of the column, coupling an input piece to the top of the column, and coupling a nosepiece to the bottom of the column (FIG. 5). In some embodiments, the mesh layer is configured to provide structural stability to the membrane layer and the column. Further, conforming the 2-ply material of the device in various three-dimensional shapes to allow for greater surface area for sperm separation for any loading volume. In some embodiments, the 2-ply material may include pleats or corrugations, further increasing surface area and enhancing sperm separation.

FIGS. 5A-E illustrate the assembly of the SSS device in some embodiments. First, the 2-ply walls are formed by aligning equal-sized rectangular-shaped sheets of the mesh layer 15 and the membrane layer 16. In some embodiments, the mesh layer 15 may be a stainless steel. In some embodiments, the membrane layer 16 may be a polycarbonate. The mesh layer 15 and membrane layer 16 may be adhered to one another using a thin bead of UV-cured acrylate along the edges of the cut materials, forming a single 2-ply sheet from the two materials. In some embodiments, conforming the 2-ply material includes curling the 2-ply material around a cylindrical form of a specific diameter and length, with the membrane layer 16 facing outward (FIG. 5B) and the mesh layer 15 facing inward, and securing the 2-ply material with a clamp, forming a cylindrical column 17 (FIG. 5C). The cylindrical shape of the 2-ply sheet may be stabilized with a thin bead of UV-cured acrylate applied longitudinally, along a region of overlap. The method further includes removal of the column 17 from the cylindrical form, and fitting the stem 7 of loading ring 1 and the stem 10 of nosepiece 9 into the opposite ends of the 2-ply column 17 (FIG. 5D). The column may be coupled to the loading ring 1 and the nosepiece 9 with a thin circumferential bead of UV-cured acrylate or epoxy (FIG. 5E); for the nosepiece 9, the bead of acrylate may overlap the “fitting ridge” 11. Other permutations of assembly may include the use of fasteners, ties, bands and/or washers, as well as heat fusion, ultrasonic welding, and/or infrared welding of membrane and/or fasteners to secure the various elements of the SSS device. In some embodiments, the 2-ply material may be formed into columns of various shapes, such as triangular columns, rectangular columns, or any other suitable shape.

In some embodiments, the loading ring 1 and the nosepiece 9 include polypropylene (FIG. 6A). Polypropylene is relatively inexpensive, easily mass-produced via injection molding, and can be heat sterilized. In some embodiments, small ball bearing(s) of galvanized steel or other material may be embedded in the nosepiece, such that the device is configured to displace a culture medium in a centrifuge tube or other vial or tube to ensure that the culture medium extends up a length of the wall of the column of the device (FIG. 1B).

In some embodiments, medical grade commodity and engineering resins with suitable density (a specific gravity greater than water) may be used for the loading ring and/or the nosepiece. In some embodiments, the loading ring and/or the nosepiece may be formed using injection molding or 3-D printing, such that the size, shape, and subtle contours of the loading ring and nose piece can be customized to optimize fit of the SSS device into many different types and brands of culture tube, vial, cuvette or other container used for containing, processing, and evaluating cells. Examples of variations of the plastic parts for the SSS device are shown in FIG. 6C, which are based on the different variable segments 12 of the nosepiece (FIG. 5). These illustrate examples enable the SSS device to readily sink to the bottom of centrifuge tube without the need for the addition of steel bearings or other weights.

The 2-ply walls of the SSS device may include an inner stainless steel filter mesh layer 15 and an outer membrane layer 16, the mesh layer 15 and the membrane layer 16 each including a plurality of microscopic openings and pores, respectively (FIG. 5A). In some embodiments, the mesh layer includes surgical-grade stainless steel. In some embodiments, the composition of stainless steel promotes adhesion of bacterial. In further embodiments, the mesh layer includes nylon, or other flexible plastics. In some embodiments, the membrane layer includes sterilized polycarbonate or polymers such as polyamide, cellulose acetate, polypiperazine-amide, or others.

FIGS. 6A-C depict devices for the collection of motile sperm, the devices include a column having a top, a bottom, and a wall including a mesh layer and a membrane layer, an inlet disposed at the top of the column, and a nosepiece disposed at the bottom of the column, according to an example embodiment. In some embodiments, the membrane layer includes a plurality of micropores. In some embodiments, the mesh layer includes a plurality of openings and is configured to restrict debris, clumps of agglutinated cells and other materials, and semen coagulum from permeating the mesh layer. In some embodiments, the micropores of the membrane layer have a diameter of about 5 μm the openings of the mesh layer have a size of about 25 μm. In some embodiments, the mesh layer includes polycarbonate and the membrane layer includes stainless steel.

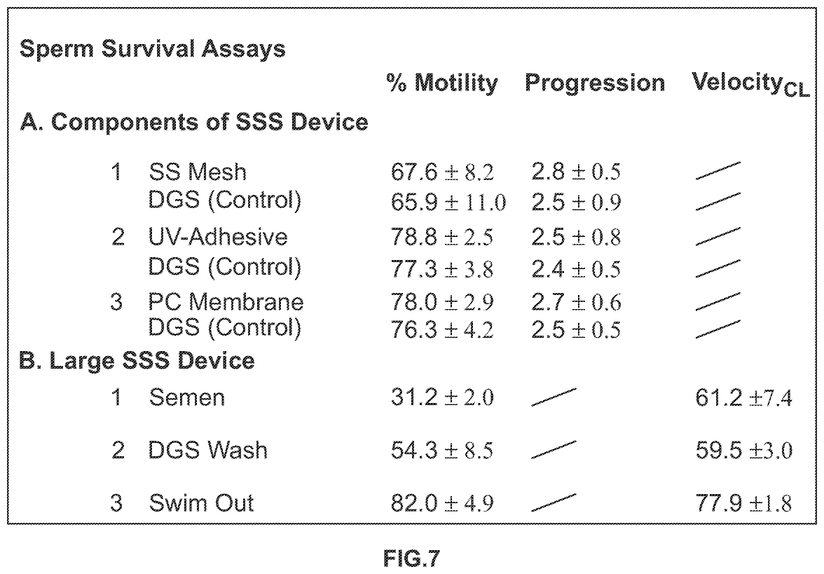

The individual elements of the device as well as the fully assembled device were tested for toxicity to sperm (FIG. 7A-B). The three components of the SSS device, the stainless-steel mesh, the polycarbonate membrane, and the UV-Cured adhesive were incubated separately with sperm washed by DGS into culture medium optimized for sperm handling (VitroLife®, Inc., Human Tubal Fluid with 10% Serum Supplement or HTF 10% S). 1 ml of washed sperm suspension adjusted to 10×10⁶ motile sperm/ml with HTF10% S were incubated continuously with the device components for 6 hours at 37 °C. There was no difference in sperm survival compared to washed sperm in controls as determined by manual motility counts of at least 400 sperm (% Motility) or the quality of progressive motility, as assessed with a 0-4 scoring system (FIG. 7A). Sperm toxicity was also determined with the fully assembled large (4 ml capacity) device (FIG. 6A). Semen was loaded into the device and sperm were allowed to swim out into HTF 10% S in a standard 15 ml centrifuge tube over 60 min. The device was removed from the centrifuge tube and the Swim Out Sperm were incubated an additional 24 hours at room temperature in the Swim Out Medium. The survival and vigor of Swim Out Sperm exceeded that of sperm in semen and sperm washed via DGS into the HTF 10% S (FIG. 7B). Viability was determined by manual motility counts of at least 400 sperm (% Motility) and vigor of sperm motility was determined using computer assisted sperm analysis (CASA; Amann and Waberski, 2014, for review) of sperm curvilinear velocity (VCL or Velocity_{CL}) (FIG. 7B). VCL values are reported in microns/sec and represent the average of motion tracks of least 500 sperm digitally captured and processed by CASA. All experiments were performed with three to four semen specimens, one from each of three to four different donors. All data in the table is reported as means+/−standard error of the mean (sem). All studies performed thereafter use the same methods of determination for both % Motility and VCL.

Initial Swim Out experiments were performed with the small SSS device (1.8 ml capacity; FIG. 6B). Preliminary tests included the evaluation of membranes of different pore sizes (5 μm and 8 μm) and stainless-steel mesh with different mesh sizes (25 μm and 50 μm). All combinations produced similar results with respect to numbers and quality of Swim Out Sperm but the combination of 5 μm pore size polycarbonate membrane and 25 μm mesh size stainless steel mesh resulted in Swim Out Medium with minimal detectable microscopic debris from seminal plasma (determined a 200× magnification). Similarly, a small series of experiments evaluated temperature on Swim Out efficiency. RT (23 °C) was compared with 30 °C and 37 °C. Higher recoveries of Swim Out Sperm were observed at both of the elevated temperatures compared to RT but no differences in Swim Out efficiency were seen between 30 °C and 37 °C. All Swim Out studies performed thereafter were conducted at 30 °C and used devices comprised of 25 μm stainless steel mesh and polycarbonate membranes with 5 μm-sized pores.

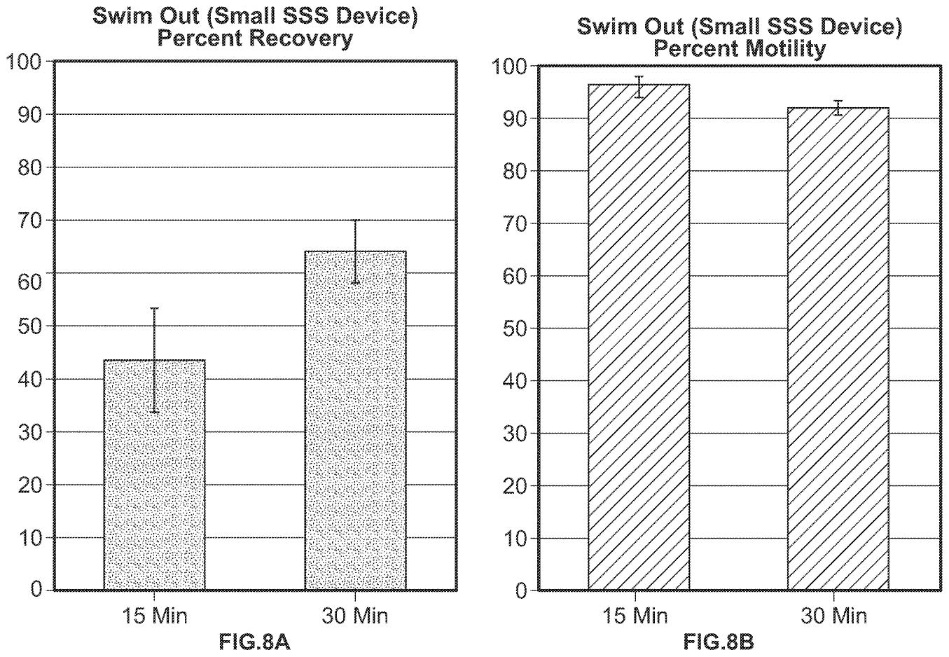

Small SSS devices were evaluated for Swim Out efficiency over 15 and 30 minutes. Semen specimens from each donor were allowed to liquefy for 30 minutes at room temperature. Two devices were loaded with 1 ml of semen from the same ejaculate. Devices were placed in cryovials containing 1 ml of HTF 10% S, one was incubated for 15 minutes and the other for 30 minutes. Observations of Swim Out times were paired and based on 12 total semen specimens, 4 specimens from each of 4 donors. On average, over 40% of the total number of motile sperm loaded into the small SSS device were recovered in Swim Out Medium after 15 minutes and nearly 65%, on average, of the total number of motile sperm loaded into the small SSS device were recovered in Swim Out Medium after 30 minutes (FIG. 8A). Columns and error bars represent means+/−sem.

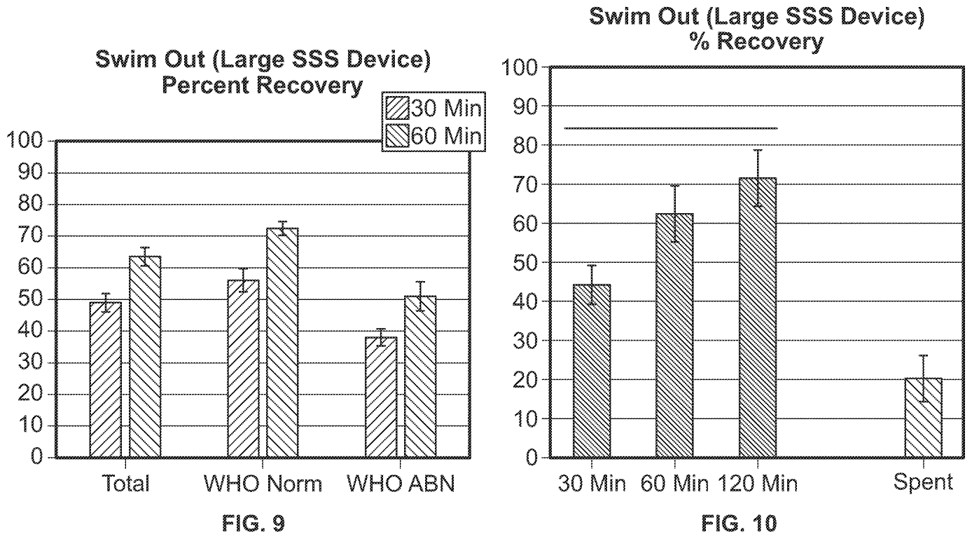

Large SSS devices were evaluated for Swim Out efficiency over 30 and 60 minutes. Semen specimens were processed almost immediately upon receipt from the donor, which was on average approximately 9 minutes following the moment of specimen collection. In most cases the specimens had not completely liquefied. Specimens were gently mixed with 0.5-1.0 ml of HTF10% S before being loaded into the SSS device. In some experiments, semen specimens were split in two, in other cases, the entire semen specimen was loaded into a single device. In no case was less than a 2.5 ml of the semen specimen used to load the SSS device. Observation of Swim Out times were not paired and based on a minimum of three to a maximum of six specimens from seven different donors. On average, the percentage of motile sperm loaded into the large SSS device that were recovered in Swim Out Medium after 30 min and 60 min was 48% and 63%, respectively (FIG. 9). Columns and error bars represent means+/−sem.

Four of the seven donors had semen specimens of consistently good quality; specimens had normal concentrations of progressively motile sperm combined with normal levels of morphologically normal sperm as determined by the 5^{th} Edition of the WHO Laboratory Manual for the Semen Analysis. By contrast, three of the seven donors consistently had poor specimen quality with lower than normal numbers of progressively motile and/or morphologically normal sperm. When the same data was stratified on the bases of WHO “Normal” (all observations from the four “good-quality” specimen donors) and WHO “Abnormal” (all observations from the three “poor-quality” specimen donors), the recovery of motile sperm appeared to be highly dependent upon overall semen quality. The recovery of WHO “Normal” specimens at 30 and 60 minutes exceeded that of WHO “Abnormal” specimens by 14% and 21% respectively. Average percent recoveries of motile sperm in Swim Out Medium for WHO “Normal” specimens at 30 and 60 minutes was 57% and 73%, respectively (FIG. 9). Columns and error bars represent means+/−sem.

In another series of experiments with the large SSS device, pre-liquefied semen (as described for experiments in FIG. 9) was loaded into a single device and then moved to new centrifuge tubes containing fresh HTF with 10% Serum Supplement at 30 min and 60 min, then allowed to incubate an additional 60 min (120 min after the loading of the SSS device; FIG. 10). Each Swim Out fraction as well as the semen remaining in the device (“spent” fraction) was evaluated for total numbers of motile sperm. The average cumulative percent recovery after 30, 60, and 120 min. and the % of total motile sperm in the spent fraction are shown in FIG. 10. Observations of Swim Out fractions were paired and based on 10 total semen specimens, 2 specimens from each of 5 donors. Columns and error bars represent means+/−sem.

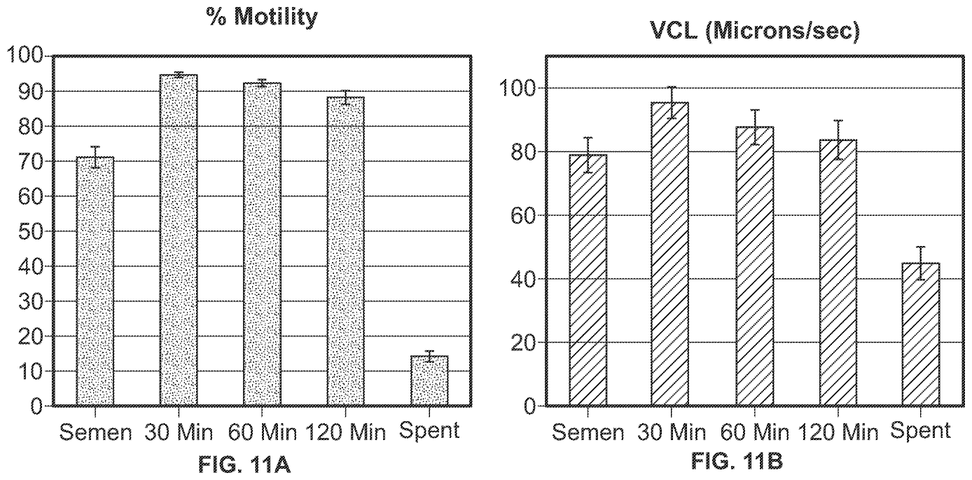

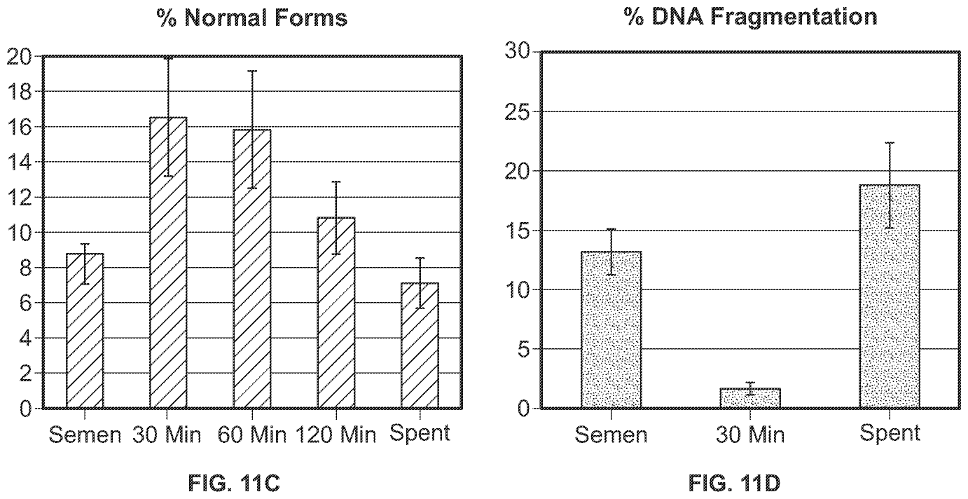

The quality of sperm in the initial semen specimen, in each Swim Out fraction, and the Spent fraction is demonstrated in FIG. 11A-D. The percentage of motile sperm recovered in the first 30 minutes of Swim Out (94.3±0.8%) was higher than that of sperm recovered in the following 30 minutes (92.1±1.1%) and in the last 60 minutes (87.9±1.9%), leaving few motile sperm remaining in the spent fraction (13.8±1.5%; FIG. 11A). Similarly, the vigor of sperm as determined by average curvilinear velocity (VCL) was highest in the initial 30-minute Swim Out fraction compared to subsequent Swim Out fractions, with all Swim Out fractions having sperm with superior VCL on average as compared to both the initial semen specimen and the spent fraction (FIG. 11B). Morphology was determined by microscope evaluation of stained sperm fixed to glass slides. A minimum of 200 sperm were evaluated from all Swim Out fractions for all experiments. Sperm classified as morphologically normal possess only minor variations in the shape and dimensions of both the flagellum and the head (Kruger et al., 1986, 1991; WHO laboratory manual, 2021). Swim Out fractions had a higher percentage of sperm with normal shape compared to either semen or the spent fraction (FIG. 11C). The TdT-mediated dUTP nick end labeling technique (TUNEL; Mochizuki, H. et al. 1994; Portera-Cailliau et. al., 1994) has been used to identify DNA damage, specifically DNA fragmentation, in spermatozoa with fluorescence microscopy (Agarwal et al., 2016; for review; Ribeiro et al., 2017). The TUNEL assay was performed in a subset of the treatments for each of the five donors. At least 200 sperm per treatment per donor were evaluated for the presence of nuclear labeling (“TUNEL-positive”) with fluorescence microscopy. The percentage of sperm selected by Swim Out with evidence of DNA fragmentation was significantly lower than that of sperm in semen and in the spent fraction (1.7±0.6%, 13.2±1.9, and 18.8±3.7, respectively; FIG. 11D). Columns and error bars represent means+/−sem.

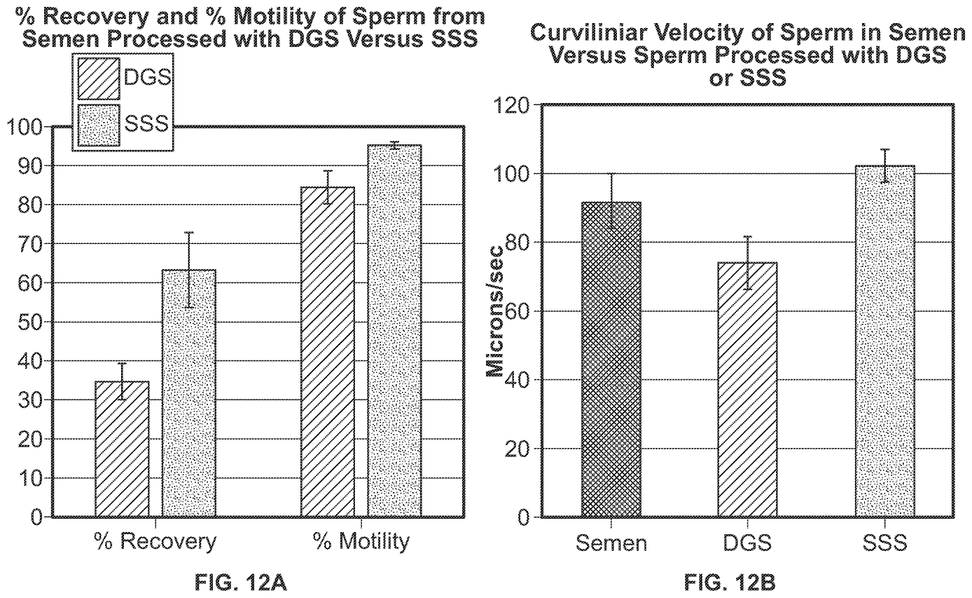

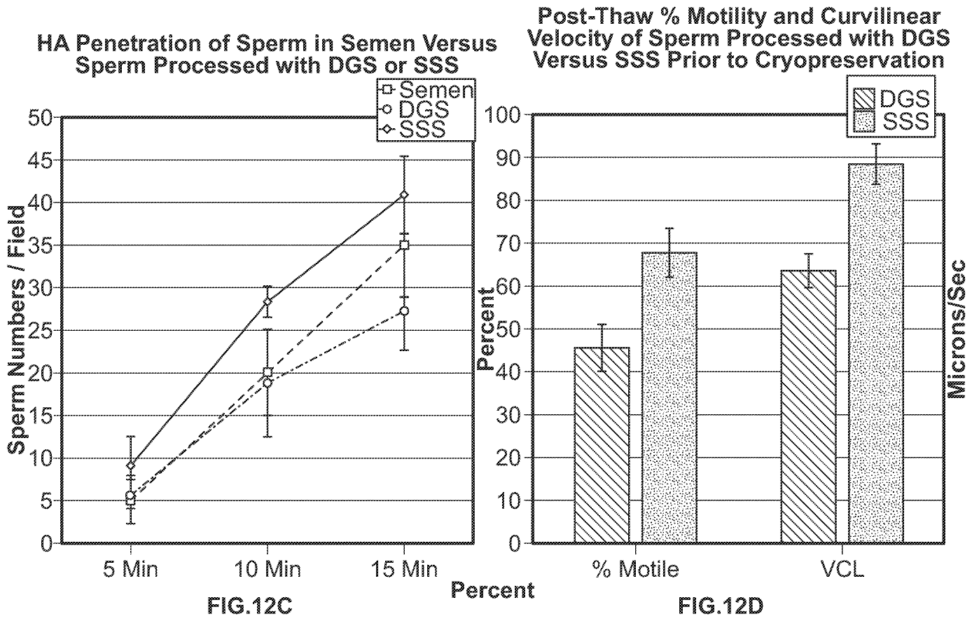

The SSS and the DGS sperm washing system were compared directly on same semen specimens for efficiency of the recovery of motile sperm and the quality of recovered sperm (FIG. 12A-D). Semen specimens were received on average 11 minutes after collection and were gently mixed with 1 ml of HTF 10% S and split into equal volumes. Half of the specimen was loaded into a large SSS device for a 60-min Swim Out, the other half was allowed to completely liquefy over the next 15-20 minutes and then loaded on top of a column of DGS (dual layer, 45/90%) in a standard 15 cc centrifuge tube. DGS washing system included an initial cycle of centrifugation of 15 min at 300×g followed by a second cycle of centrifugation with fresh HTF medium of 10 min at 300×g (to remove residual DGS; Henkel and Schill, 2003). The Swim Out medium was evaluated after 60 minutes. The split fractions of semen, one fraction loaded into large SSS device for 60 min Swim Out, the other fraction washed using DGS. The efficiency of recovery of motile sperm with the two processing methods is shown in FIG. 12A. Similar to what was observed in previous experiments, 63.1±9.8% (mean±sem) of the total number of motile sperm loaded into the SSS device were recovered in the Swim Out Medium after 60 minutes, near twice the number of motile sperm recovered with processing with DGS (34.4±4.4%; mean±sem). Experiments were performed with 4 semen specimens, one from each of four donors.

The quality of sperm separated with SSS and DGS methods is shown in FIG. 12A-D. Both the percentage of motile sperm (FIG. 12A) and the curvilinear velocity of sperm (FIG. 12B) were higher for Swim Out Sperm (sperm separated via SSS) than for sperm separated with DGS. Sperm were also evaluated for ability to penetrate gels made from long polymers of hyaluronic acid (HA). HA gels resemble cervical mucus in viscosity and charge (Aitken et al., 1992) and sperm-HA penetrations assays have been used a measure of sperm functional and structural competence (Aitken et al., 1992, 2006; Tollner et al 2008, 2011). Motile concentrations of processed sperm were adjusting to 10×10⁶/ml with HTF10% S before adding 8 ul of sperm suspension to slide chamber containing HA. Using a microscope with a grid ocular, sperm reaching a region of HA exactly 3 mm from the sperm suspension/HA interface were counted over 15 minutes. On average, penetration of HA by Swim Out Sperm (SSS) was greater than DGS-processed sperm by more than 35% (FIG. 12C). Processed sperm were frozen in vials over liquid nitrogen vapor in an egg yolk-based Tris/Tes-buffered diluent with final a final concentration of added glycerol of 5%. Frozen specimens were stored for at least 2 weeks in liquid nitrogen, then warmed for 15 min at 37 °C. The ability of Swim Out sperm to survive cryopreservation (as determined by % motility and VCL) was superior to sperm separated by DGS (FIG. 12D). Columns and error bars represent means+/−sem.

### Example SSS Devices

The SSS devices described herein provide flexibility for use with and adaptability to a broad range of culture tubes and containers used for the processing of mammalian sperm. The large device prototypes in FIGS. 6A and 6C (left) are contoured to work in a variety of makes of conical centrifuge tubes. Similarly the small device protoype in FIG. 6B works with a variety of small vials, including “cryo-vials” and “mini dispo-beakers”. Users need not purchase customized containers or chambers for collecting Swim-Out Sperm, rather they can choose from a variety of different products commonly available and of standard use in IVF and andrology laboratories. Such flexibility could accommodate laboratory preferences and have cost benefits. Furthermore, the device can be modified with respect to shape and size without altering the underlying principles and effectiveness of the design. As such, the SSS device can be adapted for use with new sperm processing systems and cultureware, including non-cyclindrical containers. The adaptability of the device lends itself to other applications, including assisted reproduction in animals, novel semen collection systems, sperm biology research, and sperm cell diagnostics (see examples).

The SSS device results in the separation and collection of sperm of superior quality from a semen specimen, leaving behind dead sperm as well as viable sperm that are likely to be damaged and non-functional. First, the sperm that exit the device are highly motile. Percent motility of Swim Out Sperm collected at 15 minutes and 30 minutes is 96.2±1.9 and 94.3±0.8 (mean±sem), respectively (FIG. 8B). These sperm that Swim Out within 30 minutes exhibit vigorous motility as determined by measures of VCL, which is on average more than double that of motile sperm that fail to exit semen in the SSS device after 2 hours (FIG. 11B). Similarly, separation through the device favors sperm with normal morphology and higher structural integrity of DNA (FIG. 11C, D). Greater than 16% of Swim Out Sperm on average possess normal shape, which is more than double the percentage of morphologically normal sperm remaining in semen (FIG. 11C). Similarly, fewer than 2% of Swim Out Sperm had detectable levels of DNA fragmentation, a level 10-fold lower than that observed for sperm remaining in semen (FIG. 11D). The quality of sperm that exit the SSS device appears to compare favorably with that reported for sperm sorted with the protoype of the Zymot® “Multi” with averages of % motility, % morphologically normal, and % DNA-fragmented of 90.4, 17.6, and 2.1, respectively (Asghar et al., 2014). Furthermore, the Swim Out duration can be extended beyond 30 minutes to increase recovery of motile sperm from semen (FIG. 10) with only a modest decrease in overall sperm quality (FIG. 11A-C).

In some embodiments, one advantage of the SSS device is the joining of a rigid yet malleable mesh to an ultra-thin, microporous membrane. The underlying (inner ply) mesh material may provide structural stability to the device and acts as a scaffold to form the overlying membrane into a number of possible 3-dimensional shapes. Various types of microporous membranes, including PCTEs, have been used for sperm separation and sperm migration studies whereby motile sperm move across a flat or planar membrane separating two chambers (Hong et al., 1991; Lee et al., 1989; Chijioke et al., 1988; Raoof et al., 1987), a configuration similar in principle to that of the Zymot® “Multi” and Lenshooke® “CA0”. By contrast, the microporous membrane of the SSS device is not constrained to a single plane or a particular shape and therefore SSS configurations may allow for greater surface area for sperm separation for any loading volume. It is conceivable with the 2-ply design to generate pleated or corrugated membrane surfaces, further increasing surface area and enhancing sperm separation.

One benefit of the SSS device is the efficiency of separation of motile sperm compared to other passive separation systems. The Zymot® “Multi”, arguably the more efficient of widely-used passive systems, has several limitations with respect to the recovery of motile sperm. First, after 30 minutes the rate of return of sperm sorted in the upper chamber (collection chamber) back into the lower chamber (loading chamber with semen) exceeds the rate of sperm movement from semen up and into the collection chamber (Asghar et al., 2014). As a result, the instructions for use of the Zymot® “Multi” suggest for best results to remove the collection medium at or before 30 minutes. The authors of (Hsu et a., 2023) make a similar recommendation for the Lenshooke® “CA0”. Without intending to be bound by theory, the present inventor believes that a simple explanation for this limitation of the “Multi” and “CA0” is that all sperm, motile and non-motile, will gradually settle with gravity to the bottom of a container, tube, or chamber, the very principle at work in migration-sedimentation systems for sorting sperm (Shany and Tavori, 2012; Kang et al., 2019; Meitei et al., 2021). Motile sperm in semen in the lower chamber of the Zymot® “Multi” and Lenshooke® “CA0” devices will eventually move away from the filter membrane and be unavailable for sorting. Second, the Zymot® “Multi” appears to have limited effectiveness with viscous samples, citing improved efficiency with 4-fold dilutions of semen specimens (Asghar et al., 2014). Third, the Zymot® only accommodates a loading volume of 850 ul, approximate ⅓ of the volume of an average ejaculate.

The SSS device may not suffer from a decrease in performance over time. Due primarily to the vertical arrangement of the filtration components of the SSS device, sperm settling with gravity presents no disadvantage and may promote higher sperm recoveries. In some embodiments, as Swim Out Sperm (those sperm outside of the device) settle, they will move below the microporous membrane region and towards the bottom of the tube where there is no possibility of exchange of sperm between Swim Out Medium and semen in the device. In such embodiments, as motile sperm inside the device settle, they will continue to move outward through the microporous walls. Support for these possibilities is given by observations in FIGS. 8 and 9 where sperm recoveries continually increase with time with no apparent differences in rates of sperm accumulation when compared to experiments where the SSS device is moved at various time intervals into tubes with fresh medium (FIG. 10).

Viscous specimens do not appear to limit the performance of the SSS device. Pre-liquefied semen specimens of varying viscosity were routinely loaded into SSS device with good results (FIGS. 9-12), owing to the ability of the stainless steal mesh of the inner ply to restrict debris and clumps of gelatinized semen from clogging pores of the outer ply polycarbonate membrane. Noteworthy for the Zymot® “Multi” and the Lenshooke® “CA0” is the requirement to let semen liquefy for 30 minutes before loading a portion of the semen specimen into the device. By contrast, semen specimens can be loaded into the SSS device shortly, if not immediately after collection. As semen liquefaction occurs gradually over 5 to 20 minutes, sperm are “released” and become more motile. The SSS device may move sperm into culture medium as soon as sperm are “available”, readily rescuing sperm from potentially toxic components of seminal plasma.

Another characteristic that distinguishes the SSS device from most sperm sorting devices in the marketplace is its ability to accommodate an entire ejaculate. From preliminary tests of materials for construction of the column, it was determined that the mesh and the microporous membrane work together to counteract the influence of hydrostatic pressure on fluid filtration. In some embodiments, there is no flow of fluid from inside the device to the outside, no matter the volume of culture medium contained in the collection tube outside of the device or volume of semen loaded inside the device. Taller devices can be devised to accommodate larger semen volumes than 4 ml. The ability to process entire ejaculate with a single device has usefulness for IUI and for the handling of specimens from species with larger average ejaculate volumes.

The separation of motile sperm using the SSS may be less harmful to sperm than DGS and may be more physiologic. In addition to demonstrating higher recoveries with the SSS device, split ejaculate studies directly comparing DGS and SSS methods show that the SSS is superior for preserving sperm quality, function and survival (FIG. 12A-D and FIG. 7B). Compared to DGS, SSS may result in the collection of a population of sperm which is more motile with respect to both percent motility and VCL (FIG. 12A). SSS-processed sperm survived better following 24-hour incubation at RT (FIG. 7B) and following freezing and thawing with standard methods of cryopreservation (FIG. 12D) than did sperm processed with DGS. After adjusting for motile sperm concentration, Swim Out Sperm and SSS-processed sperm were compared side-by-side in neighboring “micro-slide” chambers for the ability to penetrate HA gels that simulate cervical mucus. DGS resulted in lower rate of sperm-HA penetration compared to both sperm in semen and sperm processed with SSS (FIG. 12C). It has been reported previously that centrifugation of sperm with DGS alters the sperm surface by removal of sperm coating proteins and carbohydrates (Zoca et al., 2022) which greatly reduces the net negative sperm surface charge (Simon et al., 2016). Similarly, alterations of the sperm surface by enzymatic removal of negatively charged sugars or removal of the glycocalyx components via chemically induced-capacitation results in markedly reduced ability of sperm to penetrate HA gels and cervical mucus in vitro (Tollner et al., 2008, 2011). The differences in sperm-HA penetration observed here between processing methods may reflect in part a superiority of the SSS method for retaining sperm surface properties important for migration in mucus.

### EXAMPLES

The following examples are offered to illustrate, but not to limit the disclosure.

Example 1. IUI: For intrauterine insemination, it is standard practice to remove sperm from seminal plasma by “washing” sperm into culture medium by two or more rounds of centrifugation, with or without the use of density gradient solutions. The SSS device replaces these repetitive centrifugation activities. Sperm that Swim Out of the SSS device can be concentrated with a single cycle of centrifugation into a smaller volume of medium appropriate for insemination. Sperm separated with the SSS device are superior in both number and quality (% motility, VCL, and survival) than sperm separated with DGS (FIG. 12), which is important as the numbers of vigorously motile sperm per inseminate influence IUI outcomes (Ombelet et al., 2007).

Example 2. Preparation of Sperm for Cryopreservation: The SSS device can be a valuable component of the sperm processing routine for cryopreservation. Dead and dying sperm, immature sperm, and leukocytes found in semen can be sources of ROS and hydrolytic enzymes that can further damage viable sperm during the freezing and thawing process. Sperm processed with the SSS have superior post-thaw survival recovery and progressive motility compared to sperm processed with DGS (FIG. 12D).

Example 3. IVF/ICSI: Time-course studies demonstrate that sperm which exit the SSS device early on are of the highest overall quality (FIG. 8B, FIG. 11A-D). After 15 minutes, the numbers of Swim Out Sperm are more than adequate for conventional IVF and IVF with intracytoplasmic sperm injection (ICSI). The SSS can replace the Swim Up and other passive separation systems which are more difficult to set up. Therefore, incubations of semen in SSS device can be shortened to 15 min or less to obtain sperm of highest quality for IVF procedures where the numbers of motile sperm recovered are not as critical.

Example 4. Application in Research: It has been well-established in studies of mammalian reproduction that ejaculation, specifically the delivery of sperm and seminal plasma, occurs in multiple pulsatile waves resulting in various sequential fluid fractions. Up to 80% of sperm in an ejaculate are delivered in the very first 20% of the emitted fluid fractions, referred to as the “sperm rich” fractions. The remaining fluid of an ejaculate, which is primarily derived from the seminal vesicles is delivered in subsequent fractions which are rich in proteins needed to induce gelatinization of semen. During intercourse, the sperm-rich fraction is propelled into the cervix and mixes with cervical mucus. The bulk of the semen follows and forms a coagulum (a loose gelatinized plug) that is maintained in the vaginal vault, potentially blocking the cervix and helping to keep sperm from flowing back into the vagina. Sperm in the cervix gradually migrate into the upper reproductive tract. A small “wave” of highly competent sperm eventually reach the vicinity of the egg in the distal or outer oviduct, one of which will be the fertilizing sperm (Suarez and Pacey, 2006; for review; Yanagimachi, 1994; for review).

The conditions experienced by sperm when processed for either assisted reproduction or research are very different. The sperm donor collects a specimen by masturbation into a single specimen cup, resulting in mixing and extended incubation of sperm in all seminal fractions. Sperm are exposed and trapped in the coagulum, or gelatinizing fraction which greatly reduces their motility. For this reason, it is common practice to delay assessment of sperm in semen for 30 minutes, which gives sufficient time for most specimens to liquefy. Sperm remain in semen typically for 60 min while technicians assess semen for sperm motility, count, morphology, volume of semen, etc (WHO Laboratory Manual, 2010). Short term exposure to seminal plasma (30 to 60 minutes) has been shown to alter sperm function and exposure over 2 hrs hours can lead to permanent damage and death of sperm (Yavas and Selub, 2004; Punjabi et al. 2021; Iemmolo et al., 2005).

The SSS device may recover sperm from semen even before the completion of liquefaction. The SSS device can be loaded before completion of liquefaction (in most of the data presented in this application, SSS devices were loaded with semen within 11 minutes of collection). Sperm can be recovered from the device within minutes and therefore spend very little time co-incubating with seminal plasma. Preliminary studies indicate the possibility of collecting semen directly into the device with a specialized funnel and collection tube. The sperm rich fraction enters the SSS device first and sperm begin to migrate through the device and into culture medium almost instantly, avoiding direct interaction with the coagulating fractions. Physiologically speaking, these sperm would more closely resemble sperm that had entered the cervix during mating and would therefore be a better source of cells for IUI and studies of sperm biochemistry and function.

Example 5. Assisted Reproduction in Animals: Human sperm share a common form or shape with the sperm of many other mammals. Specifically, the sperm from dogs, horses, cattle, and pigs are very similar with respect to head and flagellar dimensions to that of human sperm. According to basic principle of sperm handling in humans and these species, we would expect application of the SSS device in artificial insemination and IVF technologies as they exist in animal breeding programs. For each species, changes in the mesh and membrane pore sizes may be necessary in order to optimize performance of the device but we predict that the design of the SSS devices described herein should be useful for sperm preparation in animal breeding industries.

Example 6. Patients with Poor Semen Quality: Our work with the smaller SSS device suggests that efficiency of sperm separation may increase as the device gets smaller. This stands to reason as the ratio of surface area of the polycarbonate membrane to the loading volume of semen increases with diminishing diameter of the device. Many sub-fertile men have smaller than average ejaculate volumes with fewer motile sperm. The smaller SSS device, with an apparent higher efficiency of recovery, may be better suited for use with these patients than the larger device.

Example 7. Removal of Frozen-Thawed Sperm from Cryoprotectant. Most sperm specimens frozen for long-term storage are contained in vials or straws in a total volume, including volume of added cryoprotectant, that ranges from 0.5 ml to 1.0 ml. It is common practice for fertility clinics to either wash by centrifugation or to Swim Up the frozen-thawed sperm out of cryoprotectant before using in IUI or IVF. The small SSS device could be used for this purpose, replacing these other, less efficient methods. Furthermore, the SSS device, owing to the stainless-steel mesh inner ply can be used with the more viscous cryoprotective diluents, such as those made from egg-yolk.

Example 8. In conjunction with Sperm Diagnostics: Automated male fertility diagnostics is a growing industry with numerous computer-based systems employing optical sensor/digital capture interface finding increasing acceptance in the marketplace. AI will greatly magnify the power of sperm cell analysis systems, expanding well-beyond motion analysis and morphology. We envision the use of smaller SSS devices in cuvettes, flat-sided containers with superior optical properties used for a variety of fluorometric and spectroscopic/spectrophotometric applications. Sperm separated by the SSS device will exhibit more physiologic behavior and will better retain potential surface markers of fertility compared to sperm prepared by DGS and other centrifugation methods. The efficiency of the smaller SSS device translates to a convenient means to provide sufficiently large numbers of motile sperm (the relevant sperm for fertility analysis), within minutes of loading a sperm specimen. Meanwhile, specimen “noise”, the non-viable sperm, non-spermic cells, and debris in seminal plasma, is restricted by the device and removed with the device before analysis.

Implementations of the present disclosure may thus relate to one of the enumerated example embodiments (EEEs) listed below.

EEE 1 is a device for the collection of motile sperm, the device comprising a column having a top, a bottom, and a wall, the wall comprising a mesh layer and a membrane layer, an inlet disposed at the top of the column, and a nosepiece disposed at the bottom of the column.

EEE 2 is the device of EEE 1, wherein the mesh layer is configured to structurally support the membrane layer.

EEE 3 is the device of any of EEEs 1 to 2, wherein the membrane layer comprises a plurality of micropores.

EEE 4 is the device of EEE 3, wherein the plurality of micropores includes micropores having a size of at least 1 micrometer in diameter.

EEE 5 is the device of EEE 3, wherein the plurality of micropores includes micropores having a size of less than 10 micrometers in diameter.

EEE 6 is the device of any of EEEs 1 to 5, wherein the membrane layer is configured to restrict non-motile sperm, white cells and other round cells.

EEE 7 is the device of any of EEE 1 to 6, wherein the inlet comprises a loading ring, the loading ring configured to receive liquid specimens.

EEE 8 is the device of any of EEEs 1 to 7, wherein the membrane layer comprises PVP-treated polycarbonate.

EEE 9 is the device of any of EEEs 1 to 8, wherein the column is a cylindrical column, a triangular column, or a rectangular column.

EEE 10 is the device of any of EEEs 1 to 9, wherein the mesh layer is configured to restrict debris, clumps of agglutinated cells and other material, and semen coagulum, from permeating the mesh layer.

EEE 11 is the device of any of EEEs 1 to 10, wherein the mesh layer comprises a plurality of openings, wherein the openings are at least 10 micrometers across and less than 100micrometers across.

EEE 12 is the device of any of EEEs 1 to 11, wherein the mesh layer comprises stainless steel, the stainless steel configured to restrict bacteria from permeating the mesh layer.

EEE 13 is the device of any of EEEs 1 to 12, wherein the wall further comprises a plurality of pleats or corrugations.

EEE 14 is a method for sample separation, the method comprising, receiving, by an inlet, a sample within a column comprising a mesh layer and a membrane layer according to any of EEEs 1 to 13, depositing the column in a receptacle, the receptacle containing a culture medium, capturing, by the culture medium, a portion of the sample that exits the column through the mesh layer and the membrane layer, removing the column from the receptacle, and harvesting the captured portion of the sample from the culture medium.

EEE 15 is the method of EEE 14, wherein the sample comprises liquid containing sperm.

EEE 16 is the method of EEE 15, wherein the captured portion of the sample comprises motile sperm.

EEE 17 is the method of any of EEEs 14 to 16, further comprising filtering, by the membrane layer, and adhering and filtering, by the mesh layer, at least one of debris, agglutinated cells and other material, semen coagulum, non-motile sperm, bacteria and other non-spermic cells from the sample.

EEE 18 is the method of any of EEEs 14 to 17, further comprising, depositing the column in a second receptacle, the second receptacle containing a second culture medium, and capturing, by the second culture medium, a second portion of the sample that exits the column through the mesh layer and the membrane layer.

EEE 19 is the method of any of EEEs 14 to 18, wherein the receptacle comprises a centrifuge tube, a cell-culture tube, a blood tube, a vial, or a cuvette.

EEE 20 a method for assembling a device according to any of EEEs 1 to 13, the method comprising, aligning a mesh layer and a membrane layer, adhering the mesh layer to the membrane layer, forming a two-ply material, forming, via conforming the two-ply material, a column of a predetermined diameter and length, the column having a top and a bottom, wherein the membrane layer of the two-ply material forms the outside of the column, and wherein the mesh layer of the two-ply material forms the inside of the column, coupling an input piece to the top of the column, and coupling a nosepiece to the bottom of the column.

The SSS device has been described in terms of one or more embodiments, and it should be appreciated that the many equivalents, alternatives, variations, and modifications, aside from those expressly stated, are possible and within the scope of the invention.

1. A device for the collection of motile sperm, the device comprising:
a column having a top, a bottom, and a wall, wherein the wall comprises a mesh layer and a membrane layer;
an inlet disposed at the top of the column; and
a nosepiece disposed at the bottom of the column.

2. The device of claim 1, wherein the mesh layer is configured to structurally support the membrane layer.

3. The device of claim 1, wherein the membrane layer comprises a plurality of micropores.

4. The device of claim 3, wherein the plurality of micropores includes micropores having a size of at least 1 micrometer in diameter.

5. The device of claim 3, wherein the plurality of micropores includes micropores having a size of less than 10 micrometers in diameter.

6. The device of claim 1, wherein the membrane layer is configured to restrict non-motile sperm, white cells and other round cells.

7. The device of claim 1, wherein the inlet comprises a loading ring, the loading ring configured to receive liquid specimens.

8. The device of claim 1, wherein the membrane layer comprises PVP-treated polycarbonate.

9. The device of claim 1, wherein the column is a cylindrical column, a triangular column, or a rectangular column.

10. The device of claim 1, wherein the mesh layer is configured to restrict debris, clumps of agglutinated cells and other material, and semen coagulum, from permeating the mesh layer.

11. The device of claim 1, wherein the mesh layer comprises a plurality of openings, wherein the openings are at least 10 micrometers across and less than 100 micrometers across.

12. The device of claim 1, wherein the mesh layer comprises stainless steel, the stainless steel configured to restrict bacteria from permeating the mesh layer.

13. The device of claim 1, wherein the wall further comprises a plurality of pleats or corrugations.

14. A method for sample separation, the method comprising:
receiving, by an inlet, a sample within a column comprising a mesh layer and a membrane layer;
depositing the column in a receptacle, the receptacle containing a culture medium;
capturing, by the culture medium, a portion of the sample that exits the column through the mesh layer and the membrane layer;
removing the column from the receptacle; and
harvesting the captured portion of the sample from the culture medium.

15. The method of claim 14, wherein the sample comprises liquid containing sperm.

16. The method of claim 15, wherein the captured portion of the sample comprises motile sperm.

17. The method of claim 14, further comprising filtering, by the membrane layer, and adhering and filtering, by the mesh layer, at least one of debris, agglutinated cells and other material, semen coagulum, non-motile sperm, bacteria and other non-spermic cells from the sample.

18. The method of claim 14, further comprising:
depositing the column in a second receptacle, the second receptacle containing a second culture medium; and
capturing, by the second culture medium, a second portion of the sample that exits the column through the mesh layer and the membrane layer.

19. The method of claim 14, wherein the receptacle comprises a centrifuge tube, a cell-culture tube, a blood tube, a vial, or a cuvette.

20. A method for assembling a device, the method comprising:
aligning a mesh layer and a membrane layer;
adhering the mesh layer to the membrane layer, forming a two-ply material;
forming, via conforming the two-ply material, a column of a predetermined diameter and length, the column having a top and a bottom, wherein the membrane layer of the two-ply material forms the outside of the column, and wherein the mesh layer of the two-ply material forms the inside of the column;
coupling an input piece to the top of the column; and
coupling a nosepiece to the bottom of the column.
